SEQUENCE LISTING (2 sequences)

SEQ ID NO: 1 - protein, 10 aa, Homo sapiens
DAHKSEVAHR

SEQ ID NO: 2 - protein, 10 aa, Homo sapiens
  MISC_FEATURE (5)..(5): Xaa = unknown or other
DAHKXEVAXX

(19) United States
(12) Patent Application Publication
(10) Pub. No.: US 2011/0039353 A1
(43) Pub. Date: Feb. 17, 2011

(54) METHOD FOR DIRECT DETECTION OF ISCHEMIA-MODIFIED ALBUMIN USING A PARTNER FOR BINDING TO AN ALDEHYDE DERIVATIVE RESULTING FROM THE PEROXIDATION OF LIPIDS IN BOUND FORM

(75) Inventors: Christine Des Rosiers, Montreal (CA); Colette Jolivet-Reynaud, St. Bonnet-de-Mure (FR); Jerome Martinez, Lyon (FR)

Correspondence Address: OLIFF & BERRIDGE, PLC, P.O. BOX 320850, ALEXANDRIA, VA, 22320-4850, US

(73) Assignee: BIOMERIEUX, Marcy L'Etoile (FR)

(21) Appl. No.: 12/865,562

(22) PCT Filed: Mar. 10, 2009
(86) PCT No.: PCT/FR2009/050386

§ 371 Date: Jul. 30, 2010

(30) Foreign Application Priority Data
Mar. 14, 2008 (FR) 0851648

(51) Int. Cl. G01N 33/566; G01N 33/68; C07K 16/18

(52) U.S. Cl. 436/501; 436/88; 530/388.25

(57) ABSTRACT

The present invention relates to the use of at least one partner for binding to an aldehyde derivative resulting from the peroxidation of lipids in protein-bound form, for instance to 4-hydroxy-2-nonenal, to 4-hydroxy-2-hexenal or to malondialdehyde, for the detection of ischemia-modified albumin (IMA) in a biological sample.

The present invention relates to the field of the diagnosis of ischemic conditions in humans, and in particular to a method for detection of ischemia-modified albumin (IMA).

Ischemia is the decrease in arterial blood supply to an organ. This decrease essentially causes a reduction in the oxygenation of the tissues of the organ below its needs (hypoxia), and a disruption, or even the arrest, of the function of said organ.

Ischemia can be due to a blood clot which blocks an artery (thrombosis), to an atheroma plaque, to a hemorrhage which prevents the tissues from being correctly supplied, or to compression of an artery by an external object (crushing of a limb, tourniquet) or owing to an internal phenomenon (hematoma, tumor, effusion of a liquid).

Ischemia can be reversible and cause only a limited problem. However, it can also be irreversible and result in infarction of the organ, i.e. necrosis of all or part of said organ.

IMA, which is serum albumin of which the N-terminal part is modified, is now commonly used as a marker for cardiac ischemia. In addition, a recent study has shown that IMA can also be used as a biomarker for acute stroke¹.

The detection of the presence of IMA in the blood of patients who are suspected of suffering from an ischemic condition is therefore extremely important, and those working in emergency departments seek a specific and reliable test.

At the current time, IMA is assayed indirectly by means of a colorimetric test (ACB test) which quantifies the IMA by evaluating the decrease in the capacity for cobalt ion-binding by the total albumin of the patient after modification of a part of the albumin thereof. The ACB test currently marketed has the drawbacks of not directly detecting IMA² and of detecting false positives, and it has been judged to be relatively unreliable when patients have a blood albumin level of less than 34 g/l³.

It therefore becomes urgent to have a test that overcomes the above drawbacks, in particular by assaying IMA directly.

Aldehyde derivatives resulting from the peroxidation of polyunsaturated fatty acids (lipids), which are molecules generated endogenously, such as 4-hydroxy-2-nonenal (FINE), 4-hydroxy-2-hexenal (HHE), 4-oxohexenal, 4-oxononenal and malondialdehyde (MDA), are molecules widely encountered in the body. FINE, an aldehyde of this type, is toxic and is generated by β-cleavage of hydroperoxides from ω-6 polyunsaturated fatty acids⁴. HHE also results from the peroxidation of polyunsaturated fatty acids, specifically the ω3-type fatty acids⁵. These derivatives are released in free form in biological tissues, but readily diffuse from their site of origin. Owing to their ability to bind to the nucleophilic sites of proteins and peptides, principally the histidine, lysine and cysteine residues, so as to form covalently modified biomolecules, they are therefore found either in free form, or in protein- and peptide-bound form.

Examples of proteins modified by aldehyde derivatives resulting from the peroxidation of lipids, and in particular by HNE, include hemoproteins, such as hemoglobin and myoglobin, lipoproteins such as LDLs or apolipoprotein B-100, enzymes such as glucose-6-phosphate dehydrogenase or cathepsin B⁶, and albumin which, owing to its high concentration in the serum, constitutes a preferred target for these aldehyde derivatives.

ToyoKuni S. et al.⁷ have described the importance of HNE-modified albumin for patients suffering from type 2 diabetes. Aldini G. et al.⁸ have characterized, by mass spectrometry, the covalent modification of human serum albumin (HSA) by HNE by modifying human albumin with HNE in vitro. These authors concluded, on the basis of these in vitro experiments, that this modified albumin could be useful as a biomarker in the case of patients experiencing oxidative stress, as also indicated in patent application WO2007/041868. No mention was made regarding ischemic conditions.

ToyoKuni S. et al.⁹ have described monoclonal antibodies that recognize FINE-modified bovine serum albumin and have suggested that these antibodies could be useful for evaluating the damage induced by ROSs (Reactive Oxygen Species), said ROSs being implicated in a certain number of biological phenomena such as ischemia-reperfusion. This document does not describe a marker for ischemia per se.

The present inventors have now demonstrated, against all expectations, that, in the context of ischemic conditions, ischemia-modified albumin or 1MA is in fact albumin covalently modified by a reactive aldehyde derivative resulting from the peroxidation of lipids and that it is possible to directly detect 1MA, and therefore ischemic conditions, by using this property.

Thus, a subject of the present invention is the use of a partner for binding to an aldehyde derivative resulting from the peroxidation of lipids in protein-bound form, for the detection of ischemia-modified albumin (IMA) in a biological sample.

A subject of the present invention is also a method for detection of ischemia-modified albumin (IMA) characterized in that it uses at least one partner for binding to an aldehyde derivative resulting from the peroxidation of lipids, it being understood that this aldehyde derivative recognized by the binding partner is in protein-bound form, and, when the protein for binding to said aldehyde derivative is not albumin, in that it also uses a binding partner specific for albumin.

Finally, the present invention relates to a method for in vitro diagnosis of ischemic conditions, characterized in that it implements the method for detection of IMA of the invention.

The method of the invention, which consists in detecting IMA using at least one partner for binding to an aldehyde derivative resulting from the peroxidation of lipids in protein-bound form, has the advantage that it makes it possible to detect IMA directly, thus increasing the sensitivity of detection compared with the indirect IMA detection test.

By way of example of an aldehyde derivative resulting from the peroxidation of lipids, mention may be made of 4-hydroxy-2-nonenal (HNE),4-hydroxy-2-hexenal (HHE) and malondialdehyde, which constitutes a particular embodiment of the invention.

The biological sample in which the method of the invention is carried out is any sample capable of containing IMA. By way of example of such a sample, mention may be made of blood, plasma or serum.

In order to carry out the method of the invention, consisting in using a partner for binding to the aldehyde derivative resulting from the peroxidation of lipids, it is necessary for said partner to be a partner of said derivative in protein-bound or peptide-bound form. This is because, as indicated above, the aldehyde derivatives can be found in the biological sample either in free form or in bound form, and it is desired to detect, in said sample, only said aldehyde derivatives in albumin-bound fowl. To do this, the binding partner used in the method of the invention is either specific for said derivative in albumin-bound form, or it is not specific for said aldehyde derivative in albumin-bound form, but it of course recognizes said aldehyde derivative in a form bound to another protein or peptide. In the latter case, the detection method should also use a means for isolating the albumin from the biological sample. By way of example of a means for isolating the albumin from the biological sample, mention may be made of a binding partner specific for albumin, such as an anti-human serum albumin antibody, which constitutes a particular embodiment of the invention.

According to a particular embodiment, the detection method of the invention uses both a binding partner specific for an aldehyde derivative in bound form and a binding partner specific for albumin, it being possible for said binding partner to be specific for an aldehyde derivative in albumin-bound form.

Reference is made to “binding partners specific for a molecule” when they are capable of binding to these molecules with a high specificity, or even a 100% specificity. Reference is made to “binding partners not specific for a molecule” when their specificity for binding to this molecule is low and they are then capable of binding to other ligands, such as, in the case of the aldehyde derivatives in bound form, an aldehyde derivative in a form bound to a protein other than albumin.

The method for detection of IMA of the invention can be carried out by means of any biochemical test widely known to those skilled in the art that involves molecular interactions, i.e. reactions between said aldehyde derivative resulting from the peroxidation of lipids, bound to albumin, and one or more binding partner(s) specific or not specific for same said aldehyde derivative resulting from the peroxidation of lipids.

The method for detection of IMA of the invention therefore comprises the following steps:
a biological sample capable of containing IMA is provided,
this biological sample is brought into contact with at least one binding partner for the aldehyde derivative resulting from the peroxidation of lipids in protein-bound form, and
the IMA/(binding partner for the aldehyde derivative resulting from the peroxidation of lipids in protein-bound form) binding is detected.

Of course, when the protein for said binding partner for the aldehyde derivative resulting from the peroxidation of lipids is not albumin, the method of the invention uses a partner specific for albumin so as to make it possible to isolate the albumin from said sample, as indicated above, either in detection, or in capture.

Preferably, the biochemical test is an immunoassay known to those skilled in the art that involves immunological reactions between the aldehyde derivative resulting from the peroxidation of lipids, which is the antigen, and one or more specific binding partner(s), namely the antibodies directed against this antigen.

By way of example of immunoassays as defined above, mention may be made of the sandwich methods such as ELISA, IRMA and RIA, the “competition” methods and the methods of direct immunodetection, such as immunohistochemistry, immunocytochemistry, Western blotting and dot blotting.

The binding partners specific or not specific for the aldehyde derivative(s) resulting from the peroxidation of lipids that are sought in the method of the invention, and, where appropriate, the binding partners specific for albumin, are any partner capable of binding to this or these molecule(s). By way of example, mention may be made of antibodies, antibody fractions, receptors, aptamers and any other molecule capable of binding to these molecules.

The binding-partner antibodies are, for example, either polyclonal antibodies or monoclonal antibodies.

The polyclonal antibodies can be obtained by immunization of an animal with the molecule concerned, followed by recovery of the desired antibodies in purified form, by taking a sample of the serum of said animal, and separation of said antibodies from the other serum constituents, in particular by affinity chromatography on a column to which is attached an antigen specifically recognized by the antibodies, in particular said aldehyde derivative resulting from the peroxidation of lipids.

The monoclonal antibodies can be obtained by the hybridoma technique widely known to those skilled in the art.

The monoclonal antibodies can also be recombinant antibodies obtained by genetic engineering, using techniques well known to those skilled in the art.

The antibodies against aldehyde derivatives resulting from the peroxidation of lipids in a form bound to a protein other than albumin and the anti-albumin antibodies are widely known to those skilled in the art and are sold, for example by JalCA and Hytest, respectively.

On the other hand, the antibodies which bind specifically to the aldehyde derivatives resulting from the peroxidation of lipids in albumin-bound form, whether the albumin is whole or in the form of fragments, are novel and constitute another subject of the invention.

The expression “antibodies which bind specifically to the aldehyde derivatives resulting from the peroxidation of lipids in albumin-bound form” is intended to mean any antibody capable of binding to said derivatives with a high specificity, or even a specificity of 100%, and incapable of binding to aldehyde derivatives resulting from the peroxidation of lipids in a form bound to a protein other than albumin.

According to a particular embodiment of the invention, the partner for binding to said aldehyde derivative resulting from the peroxidation of lipids in bound form is an antibody, preferably an antibody against an aldehyde derivative resulting from the peroxidation of lipids in albumin-bound form. The expression “at least one partner for binding to an aldehyde derivative from the peroxidation of lipids in bound form” is intended to mean that the method of the invention can use two or more of such partners. Thus, by way of example and in order to improve the sensitivity, the method of the invention can use a partner for binding to HNE in bound form, in particular albumin-bound form, and a partner for binding to HHE in bound form, in particular albumin-bound form, or else a partner for binding to FINE in bound form, in particular albumin-bound form, and a partner for binding to MDA in bound form, in particular albumin-bound form.

The binding partners for the aldehyde derivative resulting from the peroxidation of lipids in protein-bound form, used in the method of the invention, can be used as a capture reagent or as a detection reagent. When a binding partner specific for albumin is also used, it can be used as a capture reagent or as a detection reagent depending on whether the binding partner for the aldehyde derivative resulting from the peroxidation of lipids in protein-bound form is used, respectively, as a detection reagent or as a capture reagent.

The visualization of the immunological reactions, i.e. the IMA/binding partner binding, can be carried out by any detection means, such as direct or indirect means.

In the case of direct detection, i.e. without the intermediary of labeling, the immunological reactions are observed, for example, by surface plasmon resonance or by cyclic voltametry on an electrode bearing a conductive polymer.

The indirect detection is carried out by means of labeling, either of the binding partner termed revealing reagent, or of the IMA itself. In the latter case reference is then made to a competition method.

The term “labeling” is intended to mean the binding of a label reagent capable of directly or indirectly generating a detectable signal. A nonlimiting list of these label reagents comprises:
enzymes which produce a signal which is detectable, for example, by colorimetry, fluorescence or luminescence, such as horseradish peroxidase, alkaline phosphatase, β-galactosidase or glucose-6-phosphate dehydrogenase,
chromophores such as fluorescent, luminescent or dye compounds,
radioactive molecules such as 32P, 35S or 125I, and fluorescent molecules such as Alexa or phycocyanins.

Indirect detection systems can also be used, for instance ligands capable of reacting with an anti-ligand. Ligand/anti-ligand pairs are well known to those skilled in the art, this being the case, for example, of the following pairs: biotin/streptavidin, hapten/antibody, antigen/antibody, peptide/antibody, sugar/lectin, polynucleotide/sequence complementary to the polynucleotide. In this case, it is the ligand which carries the binding partner. The anti-ligand can be directly detectable by the label reagents described in the previous paragraph or can itself be detectable by means of a ligand/anti-ligand.

These indirect detection systems can result, under certain conditions, in an amplification of the signal. This signal amplification technique is well known to those skilled in the art, and reference may be made to prior patent applications FR98/10084 or WO-A-95/08000 by the Applicant or to the article by Chevalier et al¹⁰.

Depending on the type of labeling used, those skilled in the art will add reagents for visualizing the labeling.

Since the method of the invention makes it possible to detect ischemia-modified albumin, it is particularly suitable for the in vitro diagnosis of ischemic conditions, thereby constituting another subject of the invention.

According to a particular embodiment and in order to determine whether or not the ischemic condition is of cardiac origin, the method for in vitro diagnosis of ischemic conditions also implements the detection of a cardiac marker.

Examples of such cardiac markers comprise, without any limitation, the cardiac markers conventionally used, such as troponin, for instance troponin I or troponin T, and CK-MB (MB isoform of creatine kinase), troponin I being the preferred cardiac marker.

When, in the method of the invention, at least two markers are detected, IMA being one of them, they can be demonstrated separately, for example by means of different biochemical tests, or else simultaneously, by multiplex assay, according to the techniques previously described.

The invention will be understood more clearly by means of the following examples, given by way of nonlimiting illustration.

### EXAMPLE 1

### Edman Sequencing

The IMA was purified using its property of no longer binding divalent ions in order to isolate it from the normal HSA. For this, a pool of 10 plasmas of patients having suffered from unstable angina (ischemia) or a healthy plasma were each loaded onto a column of nickel-agarose resin (Pharmacia). Using this technique, the unmodified HSA binds to the resin, whereas the IMA is not adsorbed and passes into the filtrate. The IMA contained in the filtrates was then concentrated and immunopurified using the anti-HSA monoclonal antibody 15C7 (HyTest) coupled to a cyanogen bromide-activated Sepharose resin. After elution with a solution of 0.1 M diethylamine, pH 11.5, the IMA was isolated after SDS-PAGE electrophoresis on a 12% acrylamide gel and transferred onto a PVDF membrane. It was then sequenced using the Edman technique, with the aim of identifying whether the modification which characterizes the IMA is a cleavage of the amino acids making up its N-terminal end, as indicated in patent application WO00/20840.

Results:

| | Position | 1 | 2 | 3 | 4 | 5 | 6 | 7 | 8 | 9 | 10 |
|---|---|---|---|---|---|---|---|---|---|---|---|
| HSA: | N-term- | D | A | H | K | S | E | V | A | H | R |
| IMA: | N-term- | D | A | H | K | X | E | V | A | X | X |

For the HSA and the IMA, the same amino acids are found on the N-terminal side of the protein; the modification is not therefore a cleavage on the N-terminal side of the protein.

However, indeterminations are noted for three amino acids, which suggest that these amino acids are modified by the addition of a group.

### EXAMPLE 2

### Identification of the Albumin-Modifying Group

The SELDI-TOF technique (Ciphergen) was used to identify an addition of mass linked to the bonding of a group on the modified albumin of angina plasmas compared with the albumin of healthy samples. The interaction with the anti-hydroxynonenal antibody was then characterized with the same two angina and healthy samples.

The fractions not adsorbed onto nickel-agarose resin, of a pool of 4 angina plasmas, and also of a pool of 14 healthy sera, are immunopurified on anti-HSA resin (monoclonal antibody MAb 15C7). The eluates, enriched in IMA, derived from this double purification are analyzed by SELDI-TOF on hydrophilic arrays (NP20) and on arrays with epoxide groups (PS20) onto which are covalently grafted, via their amine function, anti-HNE antibodies (HNEJ-2, JalCA).

2-1-Analysis of the Profiles (NP20) of the Angina and Healthy Eluates

The eluates obtained after the double purification of 25 μl of the pool of angina plasmas and of the pool of healthy plasmas were diluted to 1/10 in water, and 2 μl of these solutions were deposited on an NP20 surface, as were the following three controls:
dilution to 1/10 of the eluate obtained after the double purification of an HSA-HNE (1:1) standard,
4 ng of commercial purified HSA (HSA comm.),
10 ng of HSA-HNE (1:6) standard.

After drying, the samples were analyzed by SELDI-TOF mass spectrometry (Ciphergen ProteinChip System Series 4000), with the following parameters: shots at 1500 nJ and 2000 nJ, with mass focus=66 440 Da and matrix attenuation=10 000 Da.

The results are given in table 1 below.

TABLE 1

| | | healthy pool | HSA comm. | angina pool | HSA-HNE (1:1) | HSA-HNE (1:6) |
|---|---|---|---|---|---|---|
| | | eluate (1/10) | (4 ng) | eluate (1/10) | eluate (1/10) | standard (10 ng) |
| laser | Mass read (Da) | 66621.7 | 66746.3 | 66967.2 | 67160.4 | 67334.6 |
| 1500 nj | delta mass (Da)// | 0 | 124.6 | 345.5 | 538.7 | 712.9 |
| | healthy pool | | | | | |
| laser | Mass read (Da) | 66653.8 | 66683.1 | 66893.6 | 67201 | 67415.5 |
| 2000 nj | delta mass (Da)// | 0 | 61.4 | 271.9 | 579.3 | 793.8 |
| | healthy pool | | | | | |

The results in the table demonstrate a significant shift in mass between the albumin resulting from the eluates of the pool of healthy samples and the albumin of the pool of angina samples. The albumin of the angina pool has a higher mass than the albumin of the normal samples (healthy pool eluate and commercial HSA), which means that the modification which characterizes the IMA appears to be the addition of an undefined group which leads to an increase in mass.

2-2-Analysis of the Interaction of the Angina and Healthy Eluates with the Anti-HNE Antibody (PS20)

The anti-HNE-protein monoclonal antibody HNEJ-2 (JalCA) was coupled to a PS20 surface via its epoxide groups. The eluates obtained after the double purification of 25 μl of the pool of angina plasmas, of the pool of healthy plasmas and of an HSA-HNE (1:6) standard were diluted to 1/500 in PBS (Phosphate Buffered Saline) containing 0.1% of Tween 20, and then 100 μl of this solution were brought into contact with the PS20-anti-FINE-protein MAb surface. After 3 washes in PBS-0.1% Tween, the proteins specifically retained were analyzed by SELDI-TOF mass spectrometry (Ciphergen ProteinChip System Series 4000) with the following parameters: shots at 2000 nJ, with mass focus=66 440 Da and attenuation matrix=10 000 Da.

No signal is detected for the healthy pool eluate, whereas a signal is detected for the pool of angina plasmas and for the HSA-HNE (1:6) standard. It may be concluded therefrom that the amount of HSA-HNE in the healthy samples is smaller than the amount of HSA-HNE in the angina samples.

### EXAMPLE 3

### Analysis by Gas Chromatography Mass Spectrometry

The 2 fractions not adsorbed and adsorbed onto a column of nickel-agarose resin, of the same pool of 6 angina plasmas, are immunopurified with an anti-HSA affinity resin (MAb 15C7). In this way, the IMA contained in this pool of angina plasmas in the fraction not retained by the nickel resin is concentrated.

The 2 fractions purified (IMA and HSA) are dialyzed in PBS, concentrated, quantified by OD₂₇₉ₙₘ spectrometry and stabilized by reduction with NaBD₄.

An identical amount of the IMA and of the HSA of this angina pool is analyzed by GCMS, measuring the amount of the ion corresponding to an HNE-histidine/FINE-lysine/DHN-cysteine (m/z=256) association and of the ion corresponding to the HNE-cysteine (m/z 257) association according to the method previously described¹¹.

The results are given in table 2 below.

TABLE 2

| | Signal for m/z = 256* |
|---|---|
| | (histidine-HNE, lysine-HNE, |
| Sample | cysteine-DHN) |
| IMA purified from an | 28.6 |
| angina plasma pool | |
| HSA from the same | 7.4 |
| angina plasma pool | |

*Signal for the same amount of albumin.

The results in table 2 demonstrate that the fraction of albumin not adsorbed on nickel-agarose resin, of an angina plasma, has a greater amount of HNE-histidine/HNE-lysine and DHN-cysteine groups than the fraction which binds to this nickel resin. The binding of these aldehyde derivatives to the albumin therefore leads to a loss of affinity for nickel ions and forms the IMA.

### EXAMPLE 4

### Assaying of IMA via the Assaying of HSA-HNE, HSA-HHE and HSA-MDA on a Series of “Suspicion of Acute Coronary Syndrome” Plasmas by Sandwich ELISA

Three sandwich ELISA assays are developed with the aim of assaying the hydroxynonenal, hydroxyhexenal and malondialdehyde groups bonded to albumin in the plasmas of patients, and of correlating these results with the assaying of the IMA of these same samples using the ACB test.

A solution of anti-HNE-protein monoclonal antibody (HNEJ-2 from JalCA), anti-HHE-protein monoclonal antibody (HHE53 from JalCA) or anti-MDA-protein monoclonal antibody (1F83 from JalCA), diluted to 10 μg/ml in 0.2 M Tris buffer containing 0.2 M maleic acid, at pH 6.2, was incubated for 2 h at 37° C. on a 96-well black plate (capture antibody). After saturation for 2 h at 37° C. with a solution of PBS containing 0.2% of gelatin, plasmas diluted to 1/10 in PBS-0.05% Tween were incubated overnight at 4° C. The IMA, or the HNE-modified, HHE-modified or MDA-modified albumin, specifically retained by the capture monoclonal antibody was detected by means of an anti-human serum albumin F(ab′)₂ antibody (MAb 10C3, bioMérieux, France) coupled to biotin, diluted to 1 μg/ml in PBS containing 0.05% of Tween and 0.1% of gelatin, and incubated for 2 h at 37° C. Alkaline phosphatase-coupled streptavidin diluted to 1/10 000 in TBS (Tris Buffered Saline) containing 0.05% of Tween and 0.1% of gelatin was incubated for 1 h at 37° C. A fluorometric signal measurement was then carried out after the introduction of a fluorogenic substrate for the alkaline phosphatase.

A series of 30 “suspicion of acute coronary syndrome (ACS)” plasmas, assayed beforehand using the commercial ACB test (Inverness Medical), is used in our sandwich ELISA assay. The diagnostic conclusion of these 3 tests is compared in table 3 below.

TABLE 3

| | Value | | | | | HNE + | | | |
|---|---|---|---|---|---|---|---|---|---|
| ACS | ACB | Interpretation | | | | HHE + | HNE + | HNE + | HHE + |
| plasma | (AU) | ACB | HNE | HHE | MDA | MDA | HHE | MDA | MDA |
| 642 | 46 | − | − | − | − | − | − | − | − |
| 913 | 71 | | − | − | − | − | − | − | − |
| 396 | 74 | | − | − | − | − | − | − | − |
| 901 | 74 | | − | − | − | − | − | − | − |
| 956 | 77 | + | − | + | − | + | + | − | + |
| 645 | 79 | | − | + | − | + | + | − | + |
| 941 | 80 | | − | − | − | − | − | − | − |
| 740 | 83 | | + | + | + | + | + | + | + |
| 934 | 86 | | + | + | − | + | + | + | + |
| 967 | 86 | | − | + | − | + | + | − | + |
| 613 | 87 | | − | − | − | − | − | − | − |
| 705 | 89 | | + | + | − | + | + | + | + |
| 460 | 92 | | − | + | − | + | + | − | + |
| 568 | 92 | | − | − | − | − | − | − | − |
| 979 | 92 | | − | − | − | − | − | − | − |
| 405 | 93 | | − | − | − | − | − | − | − |
| 988 | 93 | | − | + | − | + | + | − | + |
| 400 | 94 | | − | + | + | + | + | + | + |
| 921 | 94 | | + | + | − | + | + | + | + |
| 938 | 94 | | + | + | − | + | + | + | + |
| 434 | 95 | | − | + | − | + | + | − | + |
| 952 | 97 | | + | + | + | + | + | + | + |
| 565 | 99 | | − | − | − | − | − | − | − |
| 729 | 99 | | + | + | + | + | + | + | + |
| 393 | 102 | | − | + | + | + | + | + | + |
| 415 | 102 | | + | + | + | + | + | + | + |
| 946 | 102 | | − | − | − | − | − | − | − |
| 717 | 110 | | + | + | − | + | + | + | + |
| 414 | 113 | | + | + | + | + | + | + | + |
| 966 | 118 | | − | − | − | − | − | − | − |
| | | | 10 | 18 | 7 | 18 | 18 | 12 | 18 |

positives out of 26 ACB positives

The results in the table above demonstrate that the method of the invention indeed makes it possible to exclude all the non-ACS patients. On the other hand, it detects fewer patients suspected of ACS than the ACB test. This can be explained by the fact that the ACB test can give false positives. Since the final clinical diagnosis is not known, it is not certain that all the plasmas recognized as positive by the ACB test are plasmas originating from patients really suffering from ACS.

### LITERATURE

1: Aboud et al., Cerebrovasc 2007; 23: 216-220,
2: Nadhipuram V. et al., 2003, Clinical Chemistry, 49(4): 581-585
3: Gaze D C, et al., 2006, Med. Princ. Pract., 15(4): 322-324
4: Esterbauer H., et al., 1991, Free Radic. Biol. Med., 11: 81-128
5: Yamada S. et al., 2004, J. Lipid. Res., 45: 626-634
6: Carini M. et al., 2004, Mass Spectrum. Rev., 23(4): 281-305
7: ToyoKuni S. et al., 2000, Antioxidants & Redox Signaling, 2(4): 681-685
8: Aldini G. et al., 2006, Journal of Mass Spectrometry, 41: 1149-1161
9: ToyoKuni S. et al., 1995, FEBS Letters, 359: 189-191
10: Chevalier et al. 1997, J Histochem Cytochem, 45, 481-491
11: Asselin C. et al., 2006, Free Radic. Biol. Med., 41: 97-105

1. A method for the detection of ischemia-modified albumin (IMA) in a biological sample, comprising utilizing a partner for binding to an aldehyde derivative resulting from the peroxidation of lipids in protein-bound form.

2. The method as claimed in claim 1, wherein the aldehyde derivative resulting from the peroxidation of lipids is 4-hydroxy-2-nonenal, 4-hydroxy-2-hexenal or malondialdehyde.

3. The method as claimed in claim 1, wherein the at least one binding partner is specific for an aldehyde derivative resulting from the peroxidation of lipids in albumin-bound form.

4. The method as claimed in claim 1, wherein the at least one partner for binding to an aldehyde derivative derived from the peroxidation of lipids in protein-bound form is an antibody.

5. The method as claimed in claim 1, wherein the at least one partner for binding to an aldehyde derivative resulting from the peroxidation of lipids in protein-bound form is a monoclonal antibody against an aldehyde derivative derived from the peroxidation of lipids in albumin-bound form.

6. The method as claimed in claim 1, wherein it also uses a binding partner specific for albumin.

7. A method for detection of 1MA in a biological sample, wherein it uses at least one partner for binding to an aldehyde derivative resulting from the peroxidation of lipids in protein-bound form, and in that, if the protein for binding to said aldehyde derivative is not albumin, it also uses a binding partner specific for albumin.

8. The method for detection of IMA as claimed in claim 7, wherein the aldehyde derivative derived from the peroxidation of lipids is 4-hydroxy-2-nonenal, 4-hydroxy-2-hexenal or malondialdehyde.

9. The method for detection of 1MA as claimed in claim 7, wherein the at least one binding partner is specific for an aldehyde derivative resulting from the peroxidation of lipids in albumin-bound form.

10. The use method as claimed in claim 7, wherein the at least one partner for binding to an aldehyde derivative resulting from the peroxidation of lipids in protein-bound form is an antibody.

11. The method for detection of 1MA as claimed in claim 10, wherein the at least one partner for binding to an aldehyde derivative derived from the peroxidation of lipids in protein-bound form is a monoclonal antibody against an aldehyde derivative derived from the peroxidation of lipids in albumin-bound form.

12. A method for in vitro diagnosis of ischemic conditions, wherein it implements the method for detection of IMA as claimed in claim 7.

13. The method for in vitro diagnosis of ischemic conditions of the myocardium as claimed in claim 12, wherein it also implements the detection of a cardiac marker.

14. A monoclonal antibody which binds specifically to an aldehyde derivative resulting from the peroxidation of lipids in albumin-bound form.
